(19) United States
(12) Patent Application Publication
(10) Pub. No.: US 2005/0227350 A1
(43) Pub. Date: Oct. 13, 2005

(54) Device and method for studying cell migration and deformation

(75) Inventors: Manimaran Muthiah, Singapore (SG); Francis Eng-Hock Tay, Singapore (SG); Pei Lin Mao, Singapore (SG); Leonard Yi Chu, Taipei (TW)

Correspondence Address: GREG S. HOLLRIGEL, PH.D., STOUT, UXA, BUYAN & MULLINS, LLP, 4 VENTURE, SUITE 300, IRVINE, CA, 92618, US

(73) Assignee: Agency for Science, Technology and Research, Singapore (SG)

(21) Appl. No.: 10/823,164

(22) Filed: Apr. 13, 2004

(51) Int. Cl. C12M 1/34

(52) U.S. Cl. 435/288.5; 435/297.5

(57) ABSTRACT

A device for studying cell migration and deformation, a method of manufacturing and using the device. The device includes at least two channels defined therein. The two channels are separated by a partitioning wall therebetween. Each channel has an inlet and an outlet. At least one through passage is defined in the partitioning wall to allow fluid communication between the two channels. The method of manufacturing includes molding a substrate having the channels and passage defined therein and covering the substrate with a cover.

The invention relates to a device for studying at least one of cell migration and cell deformation, a method for manufacturing the device, and a method of studying at least one of cell migration and cell deformation.

### BACKGROUND OF THE INVENTION

For many years, cell motility has been a source of interest to medical researchers in various fields of medicine. In the area of inflammatory response, for example, it is well established that cell motility plays a significant role in providing healing to any injured part of the body. For example, when a stroke is sustained, leukocytes can systematically migrate out of blood capillaries into bleeding parts of the brain and assist in healing (cf. Brain Pathology 10, 127-135, 2000).

However, it is becoming clear that cell motility, although essential for survival, can also severely harm the host. Recent oncological studies suggest that cell motility may be a cause for the metastasis of cancer tumors. Metastasis refers to the spread of cancer cells from a primary tumor to distant organs within the human body. This aspect of cancer is the most devastating as it results in most cancer deaths (Keleg et al., Molecular Cancer 2, 14, 2003).

Several studies have shown that cancer cells can migrate to other parts of the body through blood vessels and lymphatic vessels (I. Carr, Cancer Metastasis, Rev. 2, 307, 1983; Yamanaka et al. Cancer Res. 53, 5289, 1993). Cancer cells have been thought to gain access into the lumen of these vessels either through existing gaps between endothelial cells of the vessels or by inducing the opening of gaps between endothelial cells.

It has also been found that angiogenesis, which refers to the growth of new blood vessels, in cancer tumors is another factor in promoting metastasis. These tumor blood vessels are believed to be leaky and have a low blood flow rate (Brown et al., Cancer Res., 58, 1408, 1998). Newly formed blood vessels within a vascularized tumor are leaky due to fact that the blood vessels lack intact endothelial cells. Cancer cells may consequently enter these blood vessels, thus bringing about the transportation of cancer cells into the blood stream. Scientists are currently exploring the opportunities for therapeutic benefit that can be derived from the intervention of cell motility during its critical stages.

The development of microfabrication and microinstrumentation techniques has gradually given rise to new methods of studying cell motility. However, these areas continue to be impeded by numerous technical challenges. Such challenges arise from considerations such as the design and implementation of necessary microfluidic functions, the integration of these functions with complete automation, and the development of cost effective manufacturing technology (M. J. Madou, Fundamentals of Microfabrication, CRC, Boca Raton, 1997).

One obstacle arises from the fact that cell invasion is an inherently hidden process occurring within a highly regulated biological system, and is thus difficult to observe and study quantitatively in vivo. While qualitative observations have been derived from traditional techniques such as intravital microscopy, they do not provide any means of quantitative measurement. In order to obtain accurate quantitative measurements, instruments that accurately simulate the in vivo cellular environment are required. Current instruments do not provide such a simulation of the in vivo cellular environment. Consequently, the quantitative aspects of cell motility within a cellular environment are not well explored.

U.S. patent application No. 2002/0168757 discloses a test device for monitoring chemotaxis, haptotaxis and chemoinvasion. The device comprises discrete chambers, each chamber having one region for holding a test agent and one region for holding a live cell sample. The two regions are connected by a canal through which the effect of the test agent on the motility of cells in the sample can be observed.

WO 03/091730 discloses a device that monitors leukocyte migration in the presence of physiological shear flow. The device comprises a well for holding leucocytes, a flow channel connected to the well, and a collection well into which effluent leukocyte is drained. In using the device, leukocytes are made to flow through the flow channel in which endothelial cells are disposed. The interaction between leukocyte and endothelial cells in the flow channel is observed.

U.S. patent application No. 2003/0030184 A1 discloses a device for arraying cells. The device comprises an array of fluid-tight micro-orifices in which cells can be deposited, and which is placed on an array of macro-orifices and studied.

Devices that involve flow dimensions that are in the micrometer range include not only those used in cell motility studies as mentioned above, but also in devices that are involved in the assaying of fluids. WO 98/21563 discloses an device for assaying small amounts of fluids. The device comprises multiple capillarity-inducing surfaces, each surface having an array of capillarity inducing structures which induces capillarity force along an axis which is perpendicular to the axis along which capillary force is induced in another region of the device.

There remains the need for biomedical instruments that provide an accurate simulation of the physiological conditions of cellular environment in the body. Consequently, it is a goal of the present invention to develop new devices for the study of cell migration and/or cell deformation. It is also a goal of this invention to provide new methods of studying cell migration and/or cell deformation which would provide the cell process and yield accurate quantitative data of different cell species that can potentially contribute to the understanding of molecular processes such as the underlying mechanisms involved in metastasis.

These goals are solved by the various aspects of the present invention, for example the device and methods as defined in the respective independent claims.

### SUMMARY OF THE INVENTION

According to an embodiment of the invention, there is provided a device for studying at least one of cell migration and deformation. The device includes at least two channels defined in the device. The two channels are separated by a partitioning wall therebetween. Each channel has an inlet and an outlet. At least one through passage is defined in the partitioning wall to allow fluid communication between the two channels.

According to another embodiment of the invention, there is provided a method of manufacturing the above-described device. The method includes providing a supporting substrate and forming a layer of photoresist on a surface of the supporting substrate. Grooves forming a pattern are photolithographically formed on the photoresist layer to define a mold. The mold is used to form a substrate of the device. The device substrate has structures forming a pattern as defined by the grooves. In other words, the mold pattern is transferred onto the substrate. Specifically, the substrate has at least two channels formed on a surface thereof, wherein the two channels are separated by a partitioning wall therebetween. Each channel has an inlet and an outlet. At least one through passage is defined in the partitioning wall to allow fluid communication between the two channels. A cover is attached to the surface of the substrate.

According to a further embodiment of the invention, there is provided a method of studying at least one of cell migration and deformation. The method comprises providing the above-described device, providing a fluid medium in at least one channel, providing a test sample containing cells in the at least second channel, and studying the migration and deformation of cells through the at least one through passage in the device.

Other aspects and advantages of the invention will become apparent from the following detailed description, taken in conjunction with the accompanying drawings, illustrating by way of example the principles of the invention.

### BRIEF DESCRIPTION OF DRAWINGS

FIG. 1 is an isometric drawing of a device according to an embodiment of the invention;

FIG. 2 is a sectional drawing of the device in FIG. 1 taken along a line AA in FIG. 1;

FIG. 3 is an isometric drawing of a device according to another embodiment of the invention;

FIG. 4 is a side elevation drawing of the device in FIG. 3 as viewed in the direction of an arrow B in FIG. 3;

FIG. 5 is an isometric drawing of a device according to yet another embodiment of the invention, having two channels separated by a partitioning wall, wherein a plurality of passages are defined in the partitioning wall;

FIG. 6 is a side elevation drawing of the device in FIG. 5 as viewed in the direction of an arrow C in FIG. 5;

FIG. 7 is a sectional drawing of the device in FIG. 5 taken along a line DD in FIG. 5;

FIG. 8 is a plan elevation drawing of the device in FIG. 5 as viewed in the direction of an arrow E in FIG. 5;

FIG. 9 is a plan elevation drawing a device similar to that in FIG. 5 showing passages of increasing widths;

FIGS. 10-14 are plan elevation drawings similar to FIG. 8 showing partitioning elements of various cross-sections that define a partitioning wall;

FIG. 15 is a side elevation drawing similar to FIG. 6 of a device having three channels;

FIG. 16 is a plan elevation drawing similar to FIG. 8 of the device in FIG. 15 as viewed in the direction of an arrow F in FIG. 15;

FIG. 17 is a plan elevation drawing similar to FIG. 9 showing a partitioning wall of increasing thickness along the length of the wall;

FIGS. 18-20 are side elevation drawings similar to FIG. 7 showing passages having cross sections of different shapes;

FIG. 21 is a plan elevation drawing of the device in FIGS. 15 and 16 showing tubes attached thereto;

FIG. 22 is a plan elevation drawing similar to FIG. 16 of a device according to a further embodiment of the invention.

FIG. 23 shows an embodiment in which two devices of the present invention are interconnected by an intervening section.

FIG. 24 shows an embodiment in which the interconnecting channels are convoluted.

FIG. 25 shows the optical microscope images taken after 2 hours of cell delivery into the device of the invention.

FIG. 26 shows the deformation and migration of cells through different passage widths of 3 μm, 5 μm and 10 μm respectively.

FIG. 27 shows the initial and later stage of the deformation of a cell through a 3 μm wide passage.

FIG. 28 shows the sequence of deformation and successful migration of a single cell through a 3 μm wide passage.

FIG. 29 shows 3 different types of cell migration through passages that are 5 μm wide.

### DETAILED DESCRIPTION OF EMBODIMENTS OF THE INVENTION

Definitions: It is understood that the terminology and definitions used herein are for the purpose of describing particular embodiments of the invention only, and not intended to be limiting.

The term “cell” as used herein refers to any type of prokaryotic or eukaryotic cell. In case of eurakyrotic cells, an assembly of cells which appear as one single cell (for example, eggs in early development) are also included in the meaning of the term “cell”. Prokaryotic cells include any type of bacteria cell such as E. coli, or bacillus subtilis or any member of the microbacterium, streptococcus or streptomyces families of bacteria. Eukaryotic cells include any type of plant or animal cell, cells such as those found in plant xylem, phloem, or meristem; or cells such as those found in animal organs, such as osteoblasts, liver cells, lung cells, stomach cells, lymphatic cells, erythrocytes and leukocytes, including monocytes and lymphocytes. Eukaryotic cells also include yeast cells. Additionally, the term includes such cells obtained from a normally functioning organ as well as those that are pathological.

The term “fluid medium” refers to any type of pure liquid or liquid solution or mixture, including water, physiologically acceptable buffers such as saline, phosphate buffer saline (PBS) or Ringer's lactate, or any type of cell media or nutrient solution such as Hank's medium or Eagle's Medium.

In one aspect, the present invention is based on microfabrication techniques, and developments in the areas of fluid mechanics and cell biology to provide a device that simulates a desired cellular environment.

The device of the invention comprises at least 2 channels. In other words, the device can have any number of channels from 2, 3, 4, 5, 6, 7, 8, 9 or 10 onwards. The number of channels that are provided may depend on several considerations, such as the flow profile, cellular throughput, or types of through passages it is desired to simulate etc. For example, if it is desired to simulate one blood vessel, it is possible to use one flow channel for delivering cells and another flow channel for delivering fluid medium. It is also possible to simulate a complex flow system by having multiple channels of more than 10 channels, some of which deliver cells and some of which deliver fluid medium.

Each channel can be of any suitable dimension. Dimensions can vary according to the requirements imposed by factors such as cell size, shear forces, type of experiment (cell-cell interaction, cell-drug interaction) and throughput etc. For example, where the intended application of the device is in the study of (small) cells such as bacterium, which typically have diameters ranging from about 0.2 to 2 μm, channels can have sizes in that range of dimensions, such as from about 0.5 to 5 μm or larger. For the study of eukaryotic cells, the dimensions of channels should be larger, since typical eukaryotic cells have diameter or sizes in the range of 10-30 μm. Leukocytes can have diameters of about 9-12 μm, while the human egg, for example, has a diameter of about 100 μm, so that the channel should accordingly be larger, e.g. 15-200 μm for these cells. Alternatively, channels can also be sized according to the dimensions of blood or lymphatic vessels e.g. blood capillaries, which typically have diameters of 20-40 μm. Furthermore, the cross-section of each channel in the present invention can be of any regular shape, such as a rectangle, square or circle. The length of each channel can be of any suitable dimension, ranging from 100 to 500 μm, or more preferably 200 to 300 μm.

The design of channel dimensions in the device can be based on considerations of the shear stress acting on the walls of the channel. Where the pressure gradient along a channel a known cross section is dP/dx, the equation for wall shear stress, τ_{w}, for a Newtonian fluid at steady state is as according to formula (I) (Drubka et al., Phys. Fluids, 10, 1723, 1989):

$${τ}_{w} = \frac{h}{2} \times \frac{ⅆ P}{ⅆ x}$$

The wall shear stress for Newtonian fluids is also determinable from flow rate Q as according to formula (II):

$${τ}_{w} = \frac{6 μ}{{h}^{2} w} \times Q$$

where w is the width of the channel and μ is the dynamic viscosity of the fluid. Based on these equations, channel dimensions can be accurately sized to simulate various types of physiological flow profiles. For example, for a given value of wall shear stress, τ_{w}, dynamic viscosity of fluid; μ, and flow rate, Q, the values of h and w can be varied to one degree of freedom. For example, to achieve a channel with a square cross-section, the width and height of the channel can have equal dimensions, i.e. w=h. Alternatively, the width can be designed to be greater than the channel height, i.e. w>>h. It is also possible that the channel width be made smaller than channel height, i.e. w<<h.

In the device of the invention, each channel has an inlet and outlet through which fluid medium and/or cells can enter and leave the device. It is possible that the inlet and outlet of each channel are similar in structure, so that fluid medium or cell can be conveniently introduced via either end of the channel. It is also possible to design the inlet and outlet differently, for example, to adapt them for secure attachment to different types of delivery tubes. The inlets and/or outlets of the channels that are designated as cell channels may also be different from the inlet and/or outlet of those channels that are designated for fluid medium. For example, the inlets and outlets of channels that would come into contact with cells can be additionally fixed with fittings that would prevent the attachment and accumulation of cells at the inlet or outlet, thus preventing any possible occlusion from taking place.

Each channel of the device communicates fluidly with an adjacent channel via one or more through passages defined in the partitioning wall. The number of through passages between any 2 adjacent channels can thus be any number from 1, 2, 3, 4, 5, 6, 7, 8, 9, 10 or greater. For example, if a small number of cells are to be studied, it may be sufficient to have one through passage through which the cells can carry out cell migration. Where large numbers of cells are studied, it is also possible to use a device with only one through passage, although the chances of occlusion occurring in the cell channel may be higher. Accordingly, it is preferable to have a larger number of through passages where large numbers of cells are studied. However, it is also possible to have a large number of through passages in the device even when a small number of cells are being studied. The number of through passages may, for example, also be dependent on the desired size of the device or the experimental value that is to be determined. If for example, the minimum width size for a cell to squeeze through is to be determined with high accuracy, it may be useful to have many through passages with small incremental changes in width size.

Each through passage may have any suitable dimension or geometry. It can be formed as a simple hole in the portioning wall or as an elongated via channel or via. Depending on the intending application, the length and width of each through passage can be varied. For example, if the device is used to study cell motility blood capillaries in which there are gaps between endothelial cells of the blood capillaries, the dimensions of the through passage can be designed to simulate the dimensions of such gaps, which are in the range of about 0.2 to 30 μm, for example. Where the motile behavior of large cells is to be studied (e.g. mammalian egg cell), the width of the through passage may be correspondingly bigger. The length of the through passage can likewise be varied. Contemplated through passage lengths may range from 50-1000 μm. If only the minimum width a particular cell type can “squeeze through” is of interest for instance, the length of the through passage can be made small. If the migration velocity is to be determined, then a longer through passage may be used. The height of the through passage can be within 10 to 60 μm, but is not limited to and thus can be smaller or larger, if desired. It is noted that it is possible to have through passages having identical or different dimensions (e.g. different width of the passage and/or different length) within the same device.

As shown in the drawings for purposes of illustrations, the invention is embodied in a device for studying cell characteristics, such as the cell-cell interaction, cell-drug interaction, migration and deformability of cancer cells, for example.

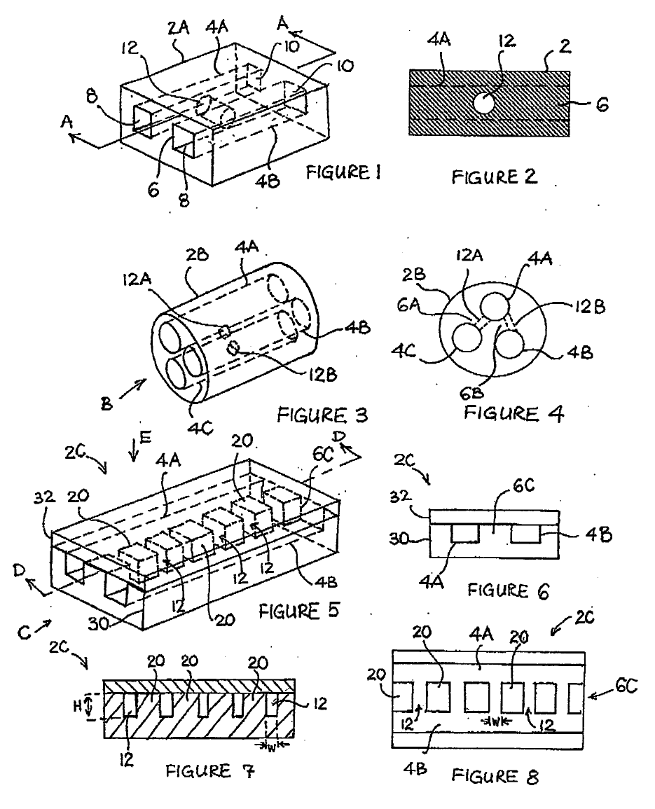

Referring to FIGS. 1 and 2, a device 2A generally includes at least two channels 4A, 4B defined therein. The two channels 4A, 4B are separated by a partitioning wall 6 therebetween. The partitioning wall 6 extends to the ends of the device 2A to define respective inlets 8 and outlets 10 of the two channels 4A, 4B. The two flow channels 4A, 4B may be straight and parallel. In other embodiments, the flow channels and the intervening through passages may be arranged along predefined curvatures, for example, in order to simulate the physiological flow in blood capillaries. For the purpose of simulating blood capillaries, it is also contemplated that the channels are arranged in an convoluted manner. At least one through via or through passage 12 is defined in the partitioning wall 6 to allow fluid communication between the two channels.

According to one embodiment, the device comprises 3 channels and two partitioning walls, each of which separate two neighbouring channels. As shown in FIGS. 3 and 4, a device 2B may include three channels 4A, 4B, 4C of a circular cross section, and two partitioning walls 6A, 6B, each of which separates two neighboring channels 4A, 4B, 4C. This embodiment provides flexibility in the location of the cell channel (i.e. the channel through which most or all of the cells are introduced into the device). For example, one of the channels can be used as the cell channel while the other 2 channels can be used as fluid medium channels. Alternatively, more than one channel can be used as cell channels. For example, it is possible to use any one of the other 2 channels as a second cell channel. It is noted that with such a configuration, both partitioning walls may be of the same or different thickness.

In another embodiment, the present device may generally include n channels and at least n-1 partitioning walls, each of which separates two neighboring channels. The channels 4A, 4B, 4C of devices that include three or more channels may be equally spaced apart as shown in FIGS. 3 and 4. However, it is contemplated that the channels can also be unequally spaced apart. For example, it is possible, in the context of FIGS. 3 and 4, that the channels 4A and 4B are arranged apart at twice the distance between 4A and 4C. Where the channels are unequally spaced apart, the effect of through passage length on cell migration can be observed. For example, it can be observed whether a through passage that is twice as long will require twice the length of time for cells to migrate across the passage.

In a further embodiment, the channels may all lie in a common plane as shown in FIG. 15. As can be seen from FIG. 1, the channels can lie in a single (i.e. one common) plane. As seen from FIGS. 3 and 4 however, channels can also lie in different planes. For example, it is possible to design a device in which the cell channel is positioned above the fluid medium channels.

In another embodiment, there are at least two passages defined in each of the at least one partitioning wall. As can be seen in FIG. 5, a device 2C includes two channels 4A and 4B separated by a partitioning wall 6C. The partitioning wall 6C is defined by a plurality of partitioning elements 20 spaced apart to form passages 12 between neighboring partitioning elements 20.

In some embodiments (not shown), the partitioning wall 6C may include two wall sections separated by a gap therebetween and at least one partitioning element 20 in the gap that divides the gap to form the two through passages 12. In one of these embodiments, at least two partitioning elements 12 are positioned in the gap between the two partitioning wall sections to divide the gap to form at least three passages 12.

Generally, such a partitioning wall may include m partitioning elements 20 in the gap that divide the gap into at least m+1 passages 12, wherein m can be any integer from I onwards. Accordingly, any one of the partitioning walls 6, 6A, 6B, 6C in the devices 2A, 2B, 2C may include two or more through passages 12 defined therein.

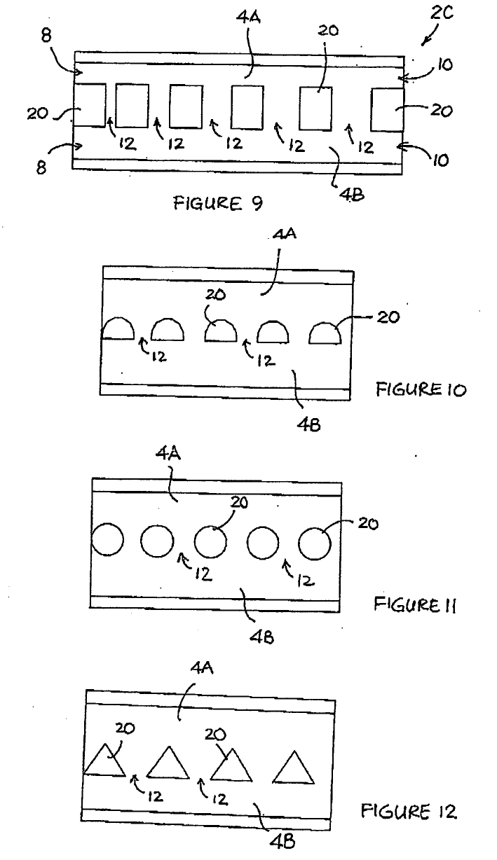

In one embodiment, the partitioning elements are at least substantially evenly spaced apart to form passages of at least substantially equal widths. For example, in the device 2C, the partitioning elements 20 are at least substantially evenly spaced apart to form passages 12 of at least substantially equal widths W, as shown in FIGS. 7 and 8. Alternatively, the partitioning elements 20 may be unevenly spaced apart, as shown in FIG. 9, to form passages 12 of widths that vary along the length of the partitioning wall 6C. In a further embodiment shown in FIG. 9, the widths of the passages 12 increases along the length of the partitioning wall. A device in which the widths of the passages increases along the length of the partitioning wall may be used for evaluating the minimum width of the through passage that is needed for a specific type of cell to migrate. In actual use, cells can be introduced into the cell channel where passage width is smallest. It is noted that cells will be unable to migrate through passages that are below cells' threshold migration size. The narrowest passage through which a cell can migrate represents the threshold/minimum migration width size. It should be noted that passages 12 of non-uniform widths might be obtained with partitioning elements of different sizes, specifically different widths, instead of spacing the partitioning elements gradually further apart.

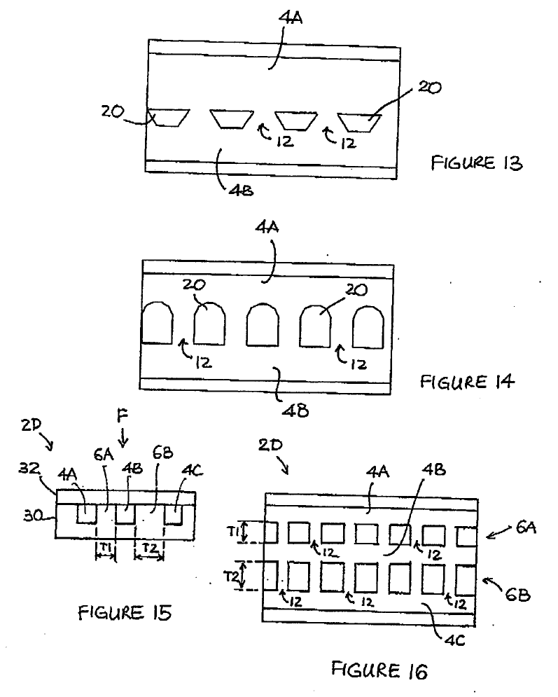

Although the partitioning elements 20 are shown in FIGS. 5, 8 and 9 to be of a rectangular cross-section, partitioning elements 20 having other cross-sections of regular or irregular shapes are also possible. Such other shapes include an oval or a trapezoid (FIG. 13). In one presently preferred embodiment, the partitioning elements 20 may have a semi-circular, circular, or triangular cross sections or an elongated cross section as shown in FIGS. 10, 11, 12, and 14 respectively. Partitioning elements of other polygonal cross-sections, such as a square, hexagonal, pentagonal, octagonal etc. and other shapes are also possible. In a further embodiment, the elongated cross-section may be rounded at least one end thereof as shown in FIG. 14.

It is also contemplated that the vertical profile of a partitioning element need not be uniform along its entire height, but can vary from the base to the top of the partitioning element. Examples of such a shape include a semi-circle, triangle, trapezium or square pyramid, or a cone. By varying the shape of the partitioning element, it is possible to modulate fluid flow profiles across the through passage, to thereby direct the flow of cells to the desired channel, thereby permitting the study of its effect on cell migration. In FIG. 10, the gradient of the semicircular partitioning element is not constant, whereas in FIG. 13, the gradient of the trapezoidal partitioning element is constant. Because of this, the strain rate of trapezium may be about 6 times greater than the semicircle. Hence it is unlikely that the cells can pass through the gap between two semicircular partitioning elements, but the cells can pass through the gap between two trapezoidal partitioning elements relatively easily due to the high strain rate. Hence, by changing the gradient i.e. consequently also the shape of, for example, endothelial cells, it is possible to avoid the cell migration through the through passages, which represent endothelial gaps of the blood vessels, for example.

In another embodiment, the thicknesses of at least one of the partitioning walls is different than that of that other partitioning walls. Referring to FIGS. 15 and 16, a device 2D includes three channels 4A, 4B, 4C with two partitioning walls 6A, 6B that separate neighboring channels 4A, 4B, 4C. The thickness T1 of one of the partitioning walls 6A is smaller than the thickness T2 of the other partitioning wall 6B. In a device with more than two partitioning walls, the thickness of one of the walls may be different than that of the other partitioning walls. Alternatively, in a further embodiment, the thicknesses of the partitioning walls are all different from each other. As shown in FIG. 17, a device 2E may have a partitioning wall 6 with a varying thickness along the length of the wall.

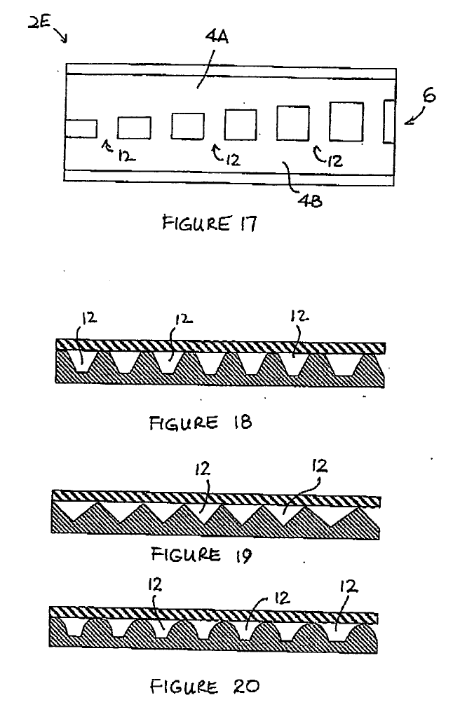

For the embodiments described above that have through passages 12 having a rectangular cross-section or opening, the rectangular cross section may have a width w and a height h as indicated in formulas (I) and (II). The width and height can have any suitable dimension. However, in the context of the invention, channel dimensions and geometry may vary, depending factors such as the sizes of cells being studied or the environment to be simulated (e.g. vein, artery, blood capillary), for example. Although dimensions of other values are also possible, in one specific embodiment, w has a dimension of between about 3-30 microns and h has a dimension of about 40 microns. The length of the through passages 12 may depend on the thickness T of the partitioning walls. In another specific embodiment, T may be in the range of about 50-100 microns. Other lengths are also possible. Cross sections of passages 12 of other shapes such as those shown in FIGS. 18, 19, and 20 are also possible.

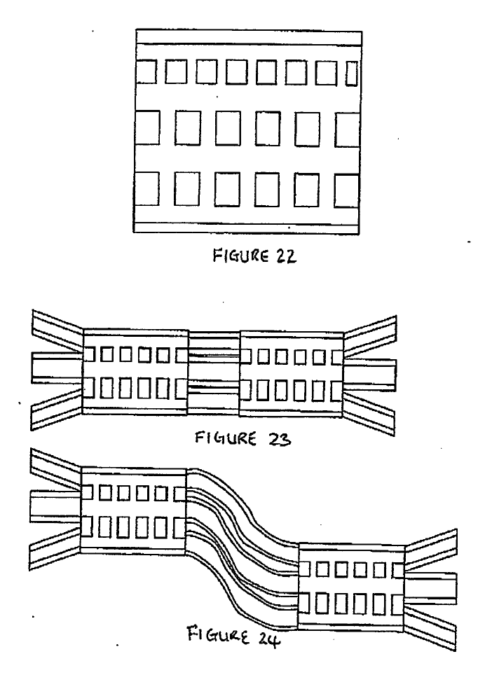

In another embodiment of the device, each outlet of a first cell migration study device is fluidly connected to the corresponding inlets of a second cell migration study device having the same number of channels as the first device, wherein the fluid connection is established via a set of interconnecting channels. As shown in FIG. 23, the interconnecting channels may be substantially straight and/or parallel. Alternatively, the interconnecting channels may be neither straight nor parallel. As shown in FIG. 24, the channels may, for example, be convoluted. The convoluted configuration permits the simulation of the physiological flow conditions in a blood vessel.

In the above embodiments, it is noted that the channels and through passages are positioned within a block of material. It is contemplated that the channels and through passages can also be formed as grooves at the surface of a block of material. In some embodiments, such as that exemplified by the devices 2C and 2D shown in FIGS. 5 and 15 respectively, the device may include a substrate 30 having a surface with grooves that define the channels 4A, 4B, 4C and the passages 12, and a cover 32 that is attached to the surface of the substrate 30. The cover may be attached for example by thermal bonding or gluing using a suitable adhesive.

The substrate may be molded using any type of material which can be made into a device of the invention and which is suitable for the observation of cells. Such materials include polymers, glass, silicone or certain types of metal. In one embodiment, the material for forming the substrate is a biocompatible material. Biocompatible material includes, but is not limited to, glass, silicon and a polymerisable material. The polymerisable material includes, but is not limited to, monomers or oligomeric building blocks (i.e. every suitable precursor molecule) of polycarbonate, polyacrylic, polyoxymethylene, polyamide, polybutylenterephthalate, polyphenylenether, polydimethylsiloxane (PDMS), mylar, polyurethane, polyvinylidene fluoride (PVDF), flourosilicone or combinations and mixtures thereof. In some embodiments, the biocompatible material comprises PVDF and/or PDMS. Advantages of PVDF and PDMS are their cheap price and superior biocompatibility. In addition, they have high gas permeability, a characteristic which is important in closed microdevices as it facilitates the permeation of supplied oxygen to the cell culture in order to ensure cell respiration. Furthermore, as they are transparent, they conveniently allow direct morphological observation of the cells under an observation device, e.g. a microscope, to be carried out.

Furthermore, the present embodiment can comprise a cover that placed on the device of the invention. The cover can have any suitable optical transparency. A fully opaque cover or one which is transparent, or one which is translucent material (thereby permitting the transmission of a certain amount of light), may all be used. In a further embodiment, the cover may comprise a biocompatible material that is transparent or at least substantially translucent in order that the device is compatible for use with optical microscopes which can provides a backlight that can be directed through the device in order to provide a bright view of the processes occurring in the device during its use.

Another aspect of the invention concerns the fabrication of the above described devices. The device of the invention can be fabricated according to any technique known in the art, such as photolithography, electron-beam lithography, laser ablation, hot embossing, etc. depending on the material used. For example, when fabricating devices using Si substrates in microscale and nanoscale, it is possible to use laser ablation or hot embossing, and electron-beam lithography respectively. The above techniques are known in the area of microelectronics and microfabrication.

Some of the devices, such as the devices shown in FIGS. 5 and 15 may be fabricated using a method of the invention as described in the following. Generally, the method comprises providing a supporting substrate, forming a layer of photoresist on a surface of the supporting substrate, and photolithographically forming grooves on the photoresist layer to define a mold. Polymerisable material is then introduced into the mold for molding the substrate 30 of the devices as described above. A cover is then attached to the surface of the substrate 30 to complete the fabrication of the device.

Where photolithography is used in the fabrication of the device, the photoresist that is utilized can be any organic polymer which becomes soluble, and thus washable, when exposed to ultraviolet light. It contains a light-sensitive substance whose properties allow image transfer onto substrates such as a PCB board. Both positive and negative photoresists may be used in the invention. Photoresists that are suitable in the present method include any commercially available photoresists such as SHIPLEY 3612, SHIPLEY S1818, or the SU-8. The SU-8 photoresist may be preferred in the invention for several reasons. The photosensitivity of SU-8 is in the range of 300-400 nm, a region accessible with conventional photolithography equipment and the high transparency in the near UV allows structures with high aspect ratios to be fabricated with near-vertical side walls. Also, because of the highly cross-linked matrix in the exposed material, it is thermally stable (up to 200° C.) and chemically stable after development. Furthermore, its solubility in a variety of organic solvents allows solutions with high solids contents to be formulated, which means that a substrate can be coated with a relatively thick film in a single spin.

More specifically, the method includes providing and washing a supporting substrate, such as a silicon wafer, with buffer oxide etchant (BOE) for about one minute. The silicon wafer is next rinsed with deionized (DI) water, dried with nitrogen gas and baked at 65° C. for about ten minutes. A layer of photoresist, such as SU-8 photoresist, is then spun on the cleaned silicon wafer at about 3000 rpm for about thirty seconds. Thereafter, the silicon wafer is relaxed at room temperature for about five minutes. Subsequently, an appropriate mask is aligned over the silicon wafer, UV exposed for about fifty seconds, and developed for about ten minutes using SU-8 developer. In this manner, grooves are lithographically formed on the photoresist layer to define a mold.

Next, the silicon wafer is then rinsed with isopropyl alcohol and DI water, dried with nitrogen gas and baked at 150° C. for about one hour. To form the substrate 30, approximately 4 g of a polymerizable material is prepared in a suitable manner to ensure that it is at least substantially bubble-free. The material is then introduced, by gently pouring it, into the mold and baked at 80° C. for about one hour until it solidifies. The solidified film, which forms the substrate 30, is then peeled off the mold. A cover 32 is then attached, for example by bonding, to an open surface of the substrate 30 to complete the fabrication of the device.

Alternatively, if laser ablation is employed, a pico-second or a femto-second laser can be directly applied onto a suitable block of any of the abovementioned materials to form grooves on the surface of the material for defining the required channels and passages, or to form channels and through passages within the material. Examples of laser ablation systems that can be used for fabricating the present device includes the LSX-3000 Nexuss from CETAC Technologies or systems from ROFIN Laser Micro, for example.

In another aspect, the present invention is directed to a method of studying cell migration and/or deformation using the device of the invention. The device of the invention can provide a way to carry out cell motility studies in a device simulating an in vivo cellular environment, thereby providing means to study processes such as cell deformation and/or cell migration.

Without wishing to be bound by theory, one possible mechanism through which cell migration may occur in vivo as well in the device of the invention consists of attachment, spreading, formation of a fine protractor, and deformation. Attachment occurs when cells flowing freely through a flow channel (e.g. blood cells flowing in a bloody capillary) attach themselves to the walls of the flow channel. After attachment, the cell spreads over the wall, and then forms fine protractors that can migrate to gaps in the wall. Deformation, aided by shearing force from flow of fluid medium, moves the cell towards and into the gap. This mechanism is repeated until the cell finally moves through the gaps.

The inventive method is carried out by simulating the above mechanism in a device of the invention, a liquid medium (e.g. a cell nutrient medium or blood plasma) and a test sample containing cells which are to be studied, are introduced into the device of the invention. By exerting hydrostatic pressure on the test sample (e.g. by elevating the cell reservoir), or by the use of an actuator such as a pump, the cells can be delivered into the cell channel. As the cells are moved through the channel(s), some cells will attach to the walls of the channel, while other cells which do not attach themselves to channel walls are washed away as the fluid medium flows out through the outlet. The attachment of cells to the channel wall marks the beginning of cell migration. Cells near to the entrance of the through passage may alter their form, e.g. by acquiring a flat shape, to enter the through passages. Some cells that are attached further away from the entrance of the through passages may also move towards the entrance of the through passages Within the through passage, cells continue to move towards the end of the through passage by extending its cytoskeleton forward. In this way, the cells finally migrate through the through passage into the adjacent channel. During the entire duration when the cells are in the device, cell migration and/or deformation can be studied in detail.

In the present method, no restriction is placed on the sequence of steps in providing the fluid medium and the test sample containing live cells. For example, it is possible to fill the device with fluid medium first, and then introduce the test sample. Alternatively, it is possible to introduce the test sample into the device, and once partially filled, the fluid medium is introduced. It is also possible to fill the device with both fluid medium and test sample simultaneously.

The flow of fluid medium in the device of the invention can be varied to simulate the effects of different types of flow on cell migration. A typical physiological shear flow condition can be in, but is not limited to, the range of about 0.1 dynes/cm² to about 20 dynes/cm². In order to simulate physiological flow conditions, the delivery of fluid and control of fluid flow in the present device can be achieved in any technique known in the art. One method is to adjust the height of the fluid medium and/or test sample reservoir. This would correspondingly adjust the hydrostatic pressure, and thus the flow rate of the fluid medium in the device. Alternatively, physiological shear flow can be provided by the use of an actuating device e.g. a pump. One or more pumps may be incorporated into the device according to any known microfabrication technique. Examples of pumps which may be used include micromachined pumps, syringe pumps, diaphragm pumps, reciprocating pumps and other pumping means known to those skilled in the art. In one embodiment, the flow rate of fluid medium is controlled by hydrostatic pressure. Hydrostatic pressure in the flow channels can be adjusted by varying the height of the reservoirs holding the cell solution or fluid medium. It is also possible to induce the flow of fluid through a channel via capillary action. Fluid flow in the device is preferably kept laminar in order to avoid any turbulence which may give rise to high shear forces leading to the damage of the cells. Such damage of cells is to be avoided since the lysis of cells may cause flow paths within the device to become clogged.

The test sample can be any type of liquid or medium containing live cells that are to be studied, such as bacteria, yeast, mammalian cells such as osteoblasts, liver cells, lung cells, stomach cells, leukocytes, erythrocytes or any type of normal or cancer etc. Such a cell may be taken from a cell line or tissue culture or extracted from a biological sample, i.e. directly isolated from the body or from an organ in the body, including fluids such as blood, plasma, semen, cervical fluid, or saliva.

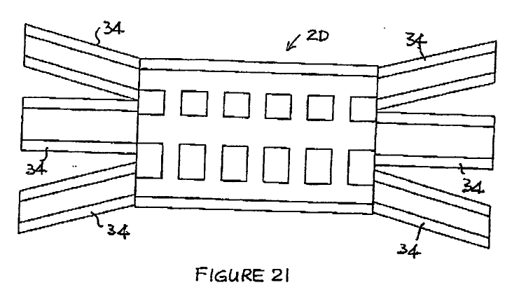

In carrying out the study of cell migration and deformation, a test sample can be introduced into the flow system through an inlet port. It is possible, for example, to transfer the cell culture into a container, e.g. a pipette, which has a flow line connected between its outlet and the inlet port of the device. As can be seen from FIG. 21, tubes 34 may be connected, such as by gluing them, to the inlets 8 and outlets 30 of the device 2D. The device 2D with attached tubes 34 is next surface-cleaned and sterilized in an oxygen plasma unit.

In one embodiment, the method further comprises filling the at least one channel with fluid medium, then allowing the flow of fluid medium to achieve steady state in all channels, and subsequently eliminating air bubbles that are present by drawing fluid medium from the outlet of the channel through which the sample containing cells is to be provided. When filling the present device with fluid medium, the introduction of air bubbles into the device is preferably avoided, as air bubbles may interfere with the flow of fluid and cell movement. If air bubbles are present, they can be eliminated prior to using the device by carrying out the following steps as follows. Firstly, the flow of fluid medium in all channels is first allowed to reach steady state. This can be observed as being the level of fluid medium in the inlets and outlets becoming the same. Once this is observed, a small amount of fluid medium, for example, a suitable amount of fluid medium is subsequently extracted from the outlet of the channel through which cells are going to be introduced. This will cause fluid medium to flow in the direction of the extracted outlet and to eliminate any air bubble present.

Cells that are to be studied in the device can be supplied in any variety of ways. For example, they can first be placed into a container, e.g. a pipette, and then allowed to flow into the device through the inlet of the channel through which cells are to be delivered. The flow rate of cells can be modulated by varying the level of fluid in the fluid medium and in the cell solution e.g. height of the reservoirs containers. Similar to the delivery of the fluid medium, it is also possible to use a pump for delivering the cells to the device.

Depending on the needs of the specific cell being studied, once the device is loaded with cells, it may or may not need to be maintained in an appropriate atmosphere. For example, if animal cells are present, the device is preferably kept in a CO₂ enriched environment at a suitable temperature. This can be provided, for example, by an incubator into which a constant stream of CO₂ is supplied. In order to keep the cells alive, cells are preferably not kept outside the incubator for more than 30 minutes. In general, mammalian cells are healthy at high CO₂ conditions, e.g. around 5% CO₂ level, but will die at atmospheric CO₂ levels. However, for other types of cells, e.g. anaerobic cells or cells that can grow under anaerobic conditions such as E. coli, an oxygen free atmosphere may be required.

Furthermore, the temperature within the device can optionally be controlled using a heater. The heater can be operatively connected to a microprocessor which controls the overall function of the system. Additionally, a means for sensing the temperature within the device may also be provided if desired.

The present device and method can be utilized in combination with an appliance for viewing cell migration. Any observation method known in the art can be utilized. For example, the motile behaviour of cells can be observed by placing the device directly under an optical microscope. The microscope can be a phase contrast, a fluorescence, luminescence, differential interference contrast, dark field, inverted, confocal laser-scanning, digital or video microscope. For example, a digital movie camera may be used to monitor cell migration over a period of time, or a camera may be used to obtain still photographic images at particular points in time. A UV light source or a laser beam can be used to activate cell fluorescence when suitable fluorescence inducing compounds are used. Other suitable optical capture devices can also be used in conjunction with the device, such as a camera, video camera, a photomultiplier tube, or a scintillation device when suitable radioactive dyes are used. It is also possible to utilize electrical conductors to enable the transmission of signals such as pressure and conductivity in the device.

The device of the invention allows quantitative measurements to be made during the study of cell migration. Parameters that can be obtained using the present device include cell velocity, the rate of migration through a unit surface area, and the minimum width permitting cell migration, for example.

One embodiment of this method comprises determining cell migration velocity in a through passage. The determination of cell migration velocity can be achieved by a variety of means. One common method is to measure the displacement of a cell at one or more time intervals. A camera may be operatively link to the device of the invention to capture images of a cell moving through the through passage at predetermined time intervals.

Another embodiment of the invention is directed to the determination of the number of cells that migrate through the at least one through passage in a specified time interval. In this embodiment, the method further comprises collecting one or more effluent fluid medium streams over a predetermined period of time, and detecting the number of cells which are present in the stream. Cells that are present in the stream can be manually counted under a microscope. One advantage here is that you can observe cell migration and count the cell number with the aid of a light microscope without the need for fluorescent labeling as it is required under in vivo study. The best way to count the cell no number is to attach a cameral recorder. If two different cells are used at the same time, then it is possible to label each cell with different fluorescence or quantum dot in order to distinguish them.

Different types of cells may exhibit different responses to different types of stimuli. In order to simulate different types of stimuli, the conditions within the device can be varied in several ways. For example, the device can be operated under a different of flow conditions, e.g. varying fluid pressure in each flow channel, or varying the velocity of flow. It is also possible to have partitioning elements with different geometries as described above to study its effect on cell migration. A further possibility is to fabricate through passages that have different aspect ratios in order to generate different flow profiles.

Surface treatment of the channels can be provided in order to improve the simulation of in vivo conditions. One possibility is to coat the inner walls of the device with cells, such as endothelial cells in order to simulate the presence of endothelial cells surrounding the cells in study. The walls of the channels and/or passages may also be coated with other compounds such as collagen or gelatin to improve biocompatibility or to simulate a certain biological environment.

In order to study the influence of cell migration and deformation mediators or cell migration and modulating (promoting or inhibiting) agents, for example the interaction between different types of cells and their counter-receptors on the endothelium, the present method can further comprise providing a cell migration promoter or inhibitor in at least one channel. These agents may comprise any biological or chemical substance, such as enzymes, proteins, hormones and pharmaceutical drugs.

Advantageously, the devices and methods that embodies the invention allows the study of migratory and growth capability of cells through at least one through passage (which may be also called micro-gap). Consequently, the device provides a good qualitative description of tumor invasion.

Although the invention is described as implemented in the above-described embodiments, it is not to be construed to be limited as such. For example, a device may have four or five channels, with neighboring channels being separated by a partitioning wall as shown in FIG. 22. The widths of the through passages of an outer wall may be smaller than that of an inner wall such that the device may function as a sieve.

### EXAMPLES

In following examples, the study of the migration, deformation and proliferation of cells through gaps ranging from 3 μm to 30 μm is carried out. The proposed device was designed using the CAD software L-edit. For mold fabrication, SU-8 2050 photoresist was used as a mold on silicon wafer for mold fabrication by photolithography. For the preparation of cell cultures, cell lines used were Dulbecco's Modified Eagle's Medium (DMEM) for C2C12 myoblast or α-Minimum Essential Medium (α-MEM) for MC3T3 osteoblast (mixed with 50 μg/ml Penicillin and 50 U/ml Streptomycin and Fetal Bovine Serum (FBS). The instruments used include a mask aligner, spin coater, plasma cleaner/sterilizer, oven, cell culture hood, centrifuge, microscope and incubator.

### Example 1

### Fabrication of a 2-Channel Microfluidic Device

1.1 Photolithography

The SU-8 photoresist was used as a mold and the fabrication process began by washing the silicon wafers with Buffer Oxide Etchant (BOE) for 1 minute. The wafers were rinsed with deionized water and dried with nitrogen gas and baked at 650C for 10 minutes. The photoresist was then spun at 3000 rpm for 30 s and was relaxed for 5 minutes at room temperature. The spun SU-8 was then soft baked first at 65° C. for 3 minutes and then at 95° C. for 10 minutes. The wafer was taken to mask aligner and UV exposed for 50 s and baked at 65° C. for 1 minute and then switched to 95° C. for 10 minutes. The wafer was cooled at room temperature for 5 minutes, then was developed for 10 min. using SU-8 Developer. The developed wafer was rinsed with isopropyl alcohol, DI water and dried with nitrogen gas and then the wafer was hard baked at 150° C. in oven for 1 hour. A SU-8 2050 mold with thickness of approximately 40 μm was fabricated.

1.2 PDMS Device Fabrication

Polydimethysiloxane (PDMS) was prepared by mixing two reagents (Silicone oil and Hardener) provided in 10:1 ratio. Approximately 4 g PDMS was prepared for each PDMS chip (each chip is about 3×2×0.3, L×W'H cm) and was vacuumed until the bubbles were invisible. The bubble-free PDMS was then poured gently onto the mold and taken to oven at 80° C. for 1 hour. Then the PDMS with the design was cut into individual devices and holes on PDMS were drilled for inlets and outlets connection. The inlets/outlets were linked to PDMS using epoxy. PDMS with epoxy was baked at 100° C. for 1˜2 hours. The baked device was taken to Plasma Cleaner/Sterilizer for surface cleaning and sterilization.

Using this method a device of the invention as shown in FIG. 21 having three channels was obtained. This device consists of 3 parallel channels each having lengths of about 300 μm, each channel separated from another by a partitioning wall, i.e. there are 2 partitioning walls. Multiple through passages (micro-gaps) present in the partitioning walls fluidly connect the central channel to the other 2 adjacent channels (FIG. 21). The width of each through passages ranges from about 3-30 μm (not shown in figure). The length of one set of through passages in one partitioning wall is 50 μm, while the length of the other set of through passages in the other partitioning wall is 100 μm. This difference in length provides two different measurements for cell migration.

1.3 Preparation of Cell Culture and Filling of the Device for Studying Cell Motility

MC3T3 osteoblasts were selected as a normal cell model for this experiment. The cells were routinely maintained in 5% CO₂ incubator at 37° C., sustained with 10% Fetal Bovine Serum in ax-Minimum Essential Medium (α-MEM) with antibiotics (penicillin/streptomycin) and the flask or petri dish contained 90% confluent cells was harvested. The old medium in the flask was completely sucked out by vacuum. To detach the cells from the flask surface, 2ml trypsin was added into the flask. After this, approx. 4 ml of α-MEM were mixed with the detached cells. The mixture of cells, trypsin and medium was then transferred to a 15 ml tube for centrifugation at the rate of 1200 rpm for 5 minutes. The supernatant was discarded and the pellet was suspended with 1 ml fresh medium. The pellet (the cells) was suspended by repeatedly pipetting. The 1 ml cell solution (approximately 10⁷ cells/ml) was transferred to a small sterile vial or eppendorf tube.

For microfluidic devices that are used in biomedical applications, such as for cell motility study, air bubbles must not appear in the channel. Air bubbles can deter or block the flow and interfere with the cell attachment and migration. Therefore, appropriate filling of the channel of the respective device with cell culture medium is a critical step. In the present experiment, the device manufactured in Example 1.2 was used. When the inlet and outlet flow of the all three channels reached equilibrium (which means the level of medium between the inlets and outlets were completely filled with the medium and the net flow rate in three channels is zero), the following optional step was carried out. 50 μl of medium were extracted out from the middle outlet. As a result, all the inlets will flow in the direction of the extracted outlet. 1 to 2 μl cell solution was delivered into the middle inlet by dipping the pipette all the way down and the flow of the cells was observed. Addition of medium to the middle outlet to balance the height difference was needed if the flow was too fast. The greater the difference of medium levels between the inlet and outlet, the faster the flow.

1.4 Observations and Discussions

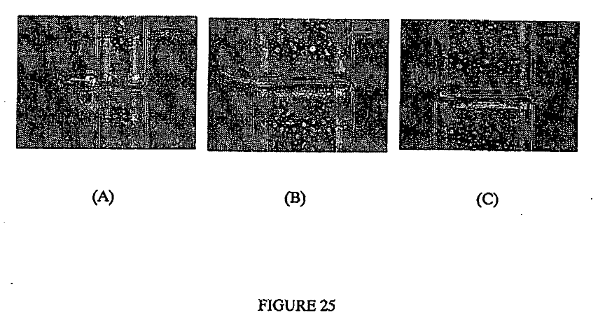

The proliferation of MC3T3 was allowed to carry on over a period of 3 days. Within the 300 μm channel, the cells were initially 60% confluent, and soon covered the entire middle channel of the device. The first set of images (FIG. 25a) was taken after 2 hours of cell delivery and the cells started to spread. The cells that are close to the through passages are of interest, because when those cells become local confluent, it is likely that the cells would spread and grow into the through passages. In FIG. 25a, the cells initially were unable to move across the 4-micron-gap. However, 7 hours later (FIG. 25b), the cells were deforming and making their ways to grow and migrate across the through passage (micro-gap) to seek more space. As shown in FIG. 25c, 24 hours later, the cells did migrate across the through passages (micro-gaps) and started proliferating.

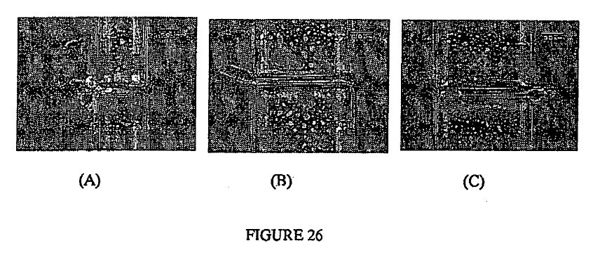

FIG. 26 shows the deformation and migration of cells through micro-gaps having different width of 3 μm, 5 μm and 10 μm, respectively. The 10 μm through passages initially were not taken because the cells were too confluent. Several hours later, due to the micro flow, the unattached cells were gradually flushed away, leaving the attached cells. At this period of time, the cells already migrated and proliferated across the 10 μm through passages.

Although cell migration might be aided by the flow, the flow was minimized when the levels of inlets and outlets were the same. So there was only a local movement of the fluid in the channel. When the cells are spherical, they are approximately 20 μm in diameter. They can easily deform themselves after attachment. As a result, the cells were unobstructed moving across the 10 μm micro-gap. As we seen from FIG. 26, the migration through the smaller gaps becomes difficult and it takes longer time to cross the through passage and to mix with the flow stream.

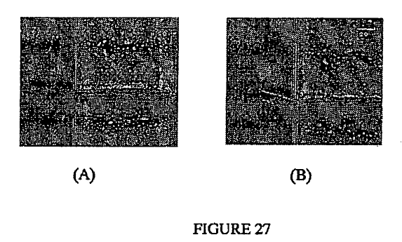

FIG. 27 shows that the nucleus of the cell itself was permanently deformed in the later stage of the migration as compared to the initial stage of the migration of cell with round nucleus. The deformed cell was traced and it was still viable even after 6 hr. This cell migrated at a rate of approximately 4 μm/hr.

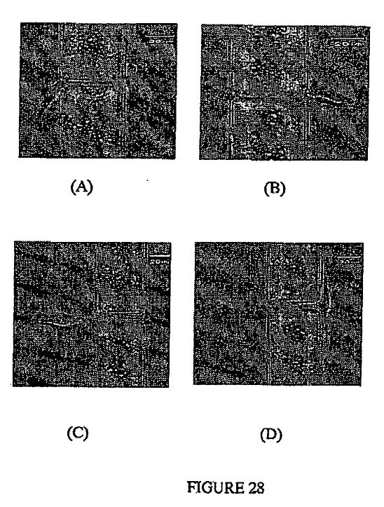

FIG. 28 shows the single cell deformation and migration of two different cells through the 3-μm through passage. In the case of FIG. 28a, the cell migrated at a speed of 7 μm/hour and in the case of FIG. 28b, the cell migrated at a speed of 11 μm/hr. Although the migration speeds were different, they all showed their capability in migrating across the through passages and surviving.

Due to the height of the through passage (40-μm), the cells may migrate in 2 dimensions through the micro-gap. However, even if this occurs, the cells still need to deform. FIG. 28 provides the evidence for deformation. In addition, due to the microfabrication limitation, the width of the through passage has an error ±1 μm. For example, a 4-micron-through passage can have a width of 3 μm or 5 μm.

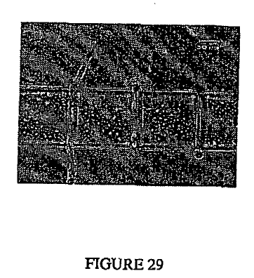

In FIG. 29 three situations are shown simultaneously. A detached spherical cell is shown to the right, which would have difficult time in passing through the micro-gap without the aid of the flow. The middle micro-gap shows two cells, one got squeezed by some flow over time and one had migrated and attached. The left micro-gap shows several attached cells spanning the mico-gap during the process of migration. Based on this observation, it is hypothesized that under a very low flow rate, the detached spherical cell cannot migrate through the micro-gaps by themselves. However, once the cells are attached, the chance of penetrating through micro-gaps is greatly enhanced. In order to move across the gap, the cells first project very thin cytoplasm across the gap, and then the nucleus gradually get “pulled” along the projected cytoplasm. This explanation is best supported by FIG. 28.

The following conclusions were drawn based on the above-described figures. The migration of MC3T3 cell is not impeded by the micro-gaps. The cells could attach to the surface and gradually migrate across the micro-gap, while deforming themselves. The deformation does not have a significant effect on the viability of cells, provided that the nutrients and gases are sufficient. According to the results, it is concluded that although the cells are deformed or sheared by flow when moving its way down the channels of the device of the invention, the deformation does not affect the viability of the cell unless it is ruptured. It is also shown that the MC3T3 cell, which is approximately 20 μm, is able to migrate through a 3 μm micro-gap. The intercellular openings of blood vessels are estimated ranging from 3 μm to 5.4 μm in diameter (Hashizume et al., 2000). These openings are the sizes that the tumor cells can potentially migrate through.

1. A device for studying at least one of cell migration and deformation comprising:
at least two channels defined in the device, the two channels being separated by a partitioning wall therebetween;
wherein each channel has an inlet and an outlet, and at least one through passage is defined in the partitioning wall to allow fluid communication between the two channels.

2. A device according to claim 1, comprising three channels and two partitioning walls, each of which separates two neighboring channels.

3. A device according to claim 1, comprising n channels and n-1 partitioning walls, each of which separates two neighboring channels.

4. A device according to claims 2 or 3, wherein the channels lie in a common plane.

5. A device according to claim 1, wherein at least two passages are defined in each of the at least one partitioning wall.

6. A device according to claim 5, wherein the partitioning wall comprises:
two wall sections separated by a gap therebetween; and
at least one partitioning element in the gap that divides the gap to form the two passages.

7. A device according to claim 6, wherein the partitioning wall comprises at least two partitioning elements that divide the gap into at least three passages.

8. A device according to claim 7, wherein the partitioning wall comprises m partitioning elements that divide the gap into at least m+1 passages.

9. A device according to claim 7, wherein the partitioning elements are at least substantially evenly spaced apart to form passages of at least substantially equal widths.

10. A device according to claim 7, wherein the partitioning elements are unevenly spaced apart to form passages of widths that vary along the length of the partitioning wall.

11. A device according to claim 10, wherein the widths of the passages increases along the length of the partitioning wall.

12. A device according to claim 7, wherein the partitioning elements have one of a semi-circular, circular, polygonal and an elongated cross section.

13. A device according to claim 12, wherein the elongated cross-section is rounded at at least one end thereof.

14. A device according to claim 2, wherein the thickness of at least one of the partitioning walls is different than that of the other partitioning walls.

15. A device according to claim 14, wherein the thicknesses of the partitioning walls are different from each other.

16. A device according to any of the preceding claims, wherein the device comprises:
a substrate whose surface has grooves that define the at least two channels and the at least one passage; and
a cover that is attached to the surface of the substrate.

17. A device according to claim 16, wherein the substrate is molded using a biocompatible material.

18. A device according to claim 17, wherein the biocompatible material is at least substantially translucent.

19. A device according to claim 16, wherein the cover is of a biocompatible material.

20. A device according to claim 19, wherein the biocompatible material is at least substantially translucent.

21. A device according to claim 17, wherein the biocompatible material comprises one of glass, silicon and a polymerizable material.

22. A device according to claim 21, wherein the polymerizable material is comprises -a material selected from the group consisting of polycarbonate (monomer), polyacrylic (monomer), polyoxymethylene (monomer), polyamide (monomer), polybutylenterephthalate (monomer) and polyphenylenether (monomer), polydimethylsiloxane (PDMS) (monomer), mylar (monomer), polyurethane (monomer), polyvinylidene fluoride (PVDF) (monomer), flourosilicone (monomer) and combinations and mixtures thereof.

23. A method of manufacturing a device for studying at least one of cell migration and deformation:
providing a substrate;
forming a layer of photoresist on a surface of the substrate;
photolithographically forming grooves on the photoresist layer to define a mold;
using the mold to mold a substrate having structures defined by the grooves, the substrate having at least two channels formed on a surface thereof, the two channels being separated by a partitioning wall therebetween, wherein each channel has an inlet and an outlet, and at least one through passage is defined in each partitioning wall to allow fluid communication between the two channels; and
attaching a cover to the surface of the substrate.

24. A method of studying at least one of cell migration and deformation, said method comprising:
providing a device comprising at least two channels defined in the device, the two channels being separated by a partitioning wall therebetween, wherein each channel has an inlet and an outlet, and at least one through passage is defined in the partitioning wall to allow fluid communication between the two channels,
providing a fluid medium in at least one channel,
providing a test sample containing cells in the at least second channel, and
studying the migration of cells through the at least one through passage.

25. The method of claim 24, wherein providing fluid medium in at least one channel comprises:
filling the at least one channel with fluid medium,
then allowing the flow of fluid medium to achieve steady state in all channels, and
subsequently eliminating air bubbles that are present by drawing fluid medium from the outlet of the channel through which the sample containing cells is to be provided.

26. The method of claims 24, wherein the flow rate of the fluid medium is controlled by hydrostatic pressure.

27. The method of claim 24, wherein the test sample is delivered to the at least second channel under laminar flow conditions.

28. The method of claim 24, further comprising determining cell migration velocity in a through passage.

29. The method of claim 24, further comprising providing a cell migration promoter or inhibitor in at least one channel.

30. The method of claim 24, wherein studying the migration of cells through the at least one through passage comprises:
observing the migration of cells by an image capture device, wherein the image capture device is a still photographic camera or a video camera.

31. The method of claim 24, further comprising determining the number of cells that migrate through the at least one through passage.
